(19) United States
(12) Patent Application Publication
(10) Pub. No.: US 2025/0012712 A1
(43) Pub. Date: Jan. 9, 2025

(54) ANALYSIS DEVICE AND ANALYSIS METHOD

(71) Applicant: HORIBA, LTD., Kyoto (JP)

(72) Inventors: Kohei TACHIBANA, Kyoto (JP); Keishi NAKAMURA, Kyoto (JP); Kodai NIINA, Kyoto (JP); Daisuke MIYAWAKI, Kyoto (JP); Tsuyoshi WATANABE, Kyoto (JP); Takuya IDO, Kyoto (JP)

(73) Assignee: HORIBA, LTD., Kyoto (JP)

(21) Appl. No.: 18/574,020

(22) PCT Filed: May 25, 2022
(86) PCT No.: PCT/JP2022/021338

§ 371 Date: Aug. 5, 2024

(30) Foreign Application Priority Data
Jun. 23, 2021 (JP) 2021-104384

(51) Int. Cl. G01N 21/31; G01N 33/00

(52) U.S. Cl. CPC G01N 21/31; G01N 33/0016; G01N 2201/0636

(57) ABSTRACT

To appropriately take explosion-proof measures for an analysis device while suppressing consumption of a purge gas, an analysis device includes a filling unit, an irradiation unit, a propagation unit, a housing, a purge gas introduction unit, and an explosion-proof gas introduction unit. The filling unit is filled with a sample gas containing a gas to be measured. The irradiation unit emits measurement light to be used for analyzing the gas to be measured. The propagation unit is disposed between the filling unit and the irradiation unit, so as to form a propagation space for propagating the measurement light emitted from the irradiation unit to the filling unit. The housing houses the filling unit, the irradiation unit, and the propagation unit. The purge gas introduction unit introduces the purge gas into the propagation space. The explosion-proof gas introduction unit introduces an explosion-proof gas into the internal space of the housing.

### CROSS-REFERENCE TO RELATED APPLICATION

This application is the U.S. national phase of PCT Application No. PCT/JP2022/021338 filed on May 25, 2022, which is incorporated herein by reference in its entirety.

### TECHNICAL FIELD

The present invention relates to an analysis device that analyzes a gas to be measured contained in a sample gas, and to an analysis method of the gas to be measured by the analysis device.

### BACKGROUND ART

Conventionally, there is known an analysis device that emits measurement light to a sample gas to analyze a gas to be measured contained in the sample gas, based on intensity of the measurement light absorbed by the gas to be measured. In this analysis device, the sample gas is filled in a predetermined cell, and the analysis is performed based on intensity of the measurement light after passing through the cell. In order to prevent the measurement light from being absorbed by gases existing in an ambient environment before reaching the predetermined cell, it is preferred to fill an optical path from a light source that emits the measurement light to the cell, with a gas containing no component that absorbs the measurement light. This gas is called a purge gas. In a measurement device for measuring carbon dioxide contained in the air, there is a method of generating a gas that does not contain the gas to be measured, by allowing the sample gas (air) to pass through a carbon dioxide adsorbent such as zeolite and a substance such as silica gel that absorbs a polar material (see, for example, JP-A-2016-14658).

In addition, there is known an apparatus that houses the analysis device in one housing. In this apparatus, explosion-proof measures are taken. Specifically, the internal space of the housing that houses the analysis device is filled with an explosion-proof gas, thereby the explosion-proof measures are taken.

### SUMMARY OF INVENTION

### Technical Problem

The gas used as the purge gas described above is usually an inert gas. Therefore, the purge gas can also be used as the explosion-proof gas that fills the internal space of the housing. However, the purge gas is more expensive than the explosion-proof gas. In addition, in order to fill the internal space of the housing with explosion-proof gas, a large amount of the gas is necessary. Since a large amount of the purge gas is necessary for filling the internal space of the housing including an optical path of the measurement light with the purge gas, an operating cost of the analysis device is increased.

On the other hand, if the internal space of the housing including the optical path of the measurement light is filled with the explosion-proof gas, the measurement light may be absorbed by a component contained in the explosion-proof gas before reaching the cell filled with the gas to be measured.

One or more embodiments of the present invention appropriately take explosion-proof measures for an analysis device while reducing consumption of a purge gas filling the optical path of the measurement light, in an apparatus that houses the analysis device for analyzing a gas to be measured in a housing.

### Technical Solution

Hereinafter, a plurality of aspects are described as means for solving the problem. These aspects can be combined arbitrarily if necessary.

An analysis device according to one aspect of the present invention is an analysis device for analyzing a gas to be measured. The analysis device includes a filling unit, an irradiation unit, a propagation unit, a housing, a purge gas introduction unit, and an explosion-proof gas introduction unit. The filling unit is filled with a sample gas containing the gas to be measured. The irradiation unit emits measurement light to be used for analyzing the gas to be measured. The propagation unit is disposed between the filling unit and the irradiation unit, to form a propagation space that is configured to propagate the measurement light emitted from the irradiation unit to the filling unit. The housing houses the filling unit, the irradiation unit, and the propagation unit. The purge gas introduction unit introduces a purge gas into the propagation space. The explosion-proof gas introduction unit introduces an explosion-proof gas into the internal space of the housing.

In the analysis device described above, the purge gas is introduced into only the propagation space as an optical path of the measurement light. As the propagation space is a space having a small volume, the consumption of the purge gas can be reduced. In addition, an explosion-proof gas different from the purge gas is introduced into the internal space of the housing having a large volume. The explosion-proof gas is inexpensive and suitable for explosion-proof measures, and hence it is possible to take explosion-proof measures for the internal space of the housing inexpensively and appropriately. In addition, by introducing the explosion-proof gas into the internal space of the housing, it is possible to cool members housed in the internal space of the housing.

The analysis device may further include a partition plate. The partition plate divides the internal space of the housing into a first internal space in which the irradiation unit and the propagation unit exist, and a second internal space in which the filling unit exists. By disposing the partition plate that divides the internal space of the housing into a first internal space in which the irradiation unit and the propagation unit exist, and a second internal space in which the filling unit exists, the irradiation unit and the propagation unit can be spatially separated from the filling unit that becomes high temperature.

The housing and the propagation unit may be constituted as an internal pressure explosion-proof container. In this way, the internal spaces of the housing and the propagation unit can be safely protected from explosion.

The analysis device may further include a pressure switch. The pressure switch detects whether or not the pressure in the filling unit becomes equal or more than a predetermined pressure lower than the pressure in the internal space of the housing. In this way, it can be determined whether or not the pressure in the filling unit has become higher than the internal space of the housing so that the gas filled in the filling unit may leak to the internal space of the housing.

The analysis device may further include a first differential pressure gauge. The first differential pressure gauge measures a difference between the pressure adjacent to the purge gas outlet in the propagation space and the pressure in the internal space of the housing. In this way, it can be securely detected whether or not the pressure in the propagation space is higher than the pressure in the internal space of the housing, i.e., whether or not the explosion-proof gas might enter the propagation space.

The analysis device may further include a second differential pressure gauge. The second differential pressure gauge measures a difference between the pressure adjacent to an explosion-proof gas outlet in the internal space of the housing and the pressure outside the housing. In this way, it can be securely detected whether or not the pressure in the internal space of the housing is higher than the pressure outside the housing, i.e., whether or not an external gas might enter the internal space of the housing so that the explosion-proof measures can be inappropriate.

The propagation unit may include a mirror disposed in the propagation space to guide the measurement light to the filling unit. In this case, the analysis device may further include a jig. The jig allows a tool that adjusts the mirror to reach a position of the mirror in the propagation space. In this way, even if the mirror cannot be visually checked, the tool that adjusts the mirror can reach an appropriate position.

The analysis device may further include a fixing plate. The fixing plate fixes the filling unit, the irradiation unit, and the propagation unit. In this way, it is possible to prevent a change in relative positions or orientations among the filling unit, the irradiation unit, and the propagation unit, due to deformation or the like of the housing.

The analysis device may further include a separating unit. The separating unit separates the purge gas from a gas. In this way, the purge gas can be produced easily and inexpensively.

The separating unit may be disposed outside the housing. In this way, it is possible to prevent components other than the purge gas contained in a gas from being discharged into the internal space of the housing.

The separating unit may be disposed in the housing. In this case, it may be possible to use a remnant gas other than the purge gas among components generated by separation of the gas using the separating unit, as the explosion-proof gas. In this way, it is not necessary to separately dispose a gas line for supplying the purge gas and a gas line for supplying the explosion-proof gas.

The analysis device may further include the first differential pressure gauge, the second differential pressure gauge, and the pressure switch. The first differential pressure gauge measures a difference between the pressure adjacent to the purge gas outlet in the propagation space and the pressure in the internal space of the housing. The second differential pressure gauge measures a difference between the pressure adjacent to the explosion-proof gas outlet in the internal space of the housing and the pressure outside the housing. The pressure switch detects whether or not the pressure in the filling unit becomes equal to or more than a predetermined pressure lower than the pressure in the internal space of the housing. In this case, the first differential pressure gauge, the second differential pressure gauge, and the pressure switch measure a magnitude relationship between the pressure in the internal space of the housing and the pressure outside the housing, a magnitude relationship between the pressure in the propagation space and the pressure in the internal space of the housing, a magnitude relationship between the pressure in the propagation space and the pressure in the internal space of the filling unit, and a magnitude relationship between the pressure in the internal space of the housing and the pressure in the internal space of the filling unit. In this way, a lot of magnitude relationships between places can be efficiently measured by a small number of devices.

The gas to be measured is carbon dioxide (CO₂), carbon monoxide (CO), methane (CH₄), sulfur dioxide (SO₂), ammonia (NH₃), nitrogen oxides (NOx), hydrogen chloride (HCl), water (H₂O), ethane (C₂H₆), acetylene (C₂H₂), propane (C₃H₈), ethylene (C₂H₄), hexane (n-C₆H₁₄), propylene (C₃H₆), hydrogen sulfide (H₂S), isobutene (i-C₄H₈), methanol (CH₃OH), phosgene (COCl₂), butane (n-C₄H₁₀), chloroethylene (C₂H₃Cl), methyl nitrite (CH₃ONO), cyclohexane (C₆H₁₂), butadiene (C₄H₆), isobutane (i-C₄H₁₀), isopentane (i-C₅H₁₂), toluene (C₆H₅CH₃), hydrogen (H₂), hydrogen fluoride (HF), or trifluoropropene (C₃H₃F₃). In this way, in the analysis device, it is possible to appropriately take explosion-proof measures to safely analyze the gas to be measured, while suppressing consumption of the purge gas.

An analysis method according to another aspect of the present invention is a method for analyzing a gas to be measured by an analysis device. The analysis device includes a filling unit in which a sample gas containing a gas to be measured is filled, an irradiation unit that emits measurement light to be used for analyzing the gas to be measured, a propagation unit disposed between the filling unit and the irradiation unit, to form a propagation space that propagates the measurement light emitted from the irradiation unit to the filling unit, and a housing that houses the filling unit, the irradiation unit, and the propagation unit. The analysis method includes:
introducing a purge gas into the propagation space;
introducing an explosion-proof gas into the internal space of the housing;
emitting the measurement light from the irradiation unit, to propagate the measurement light through the propagation space to the filling unit that is filled with the sample gas; and
analyzing the gas to be measured contained in the sample gas, based on a measurement result of the measurement light after passing through the sample gas filled in the filling unit.

In the analysis method described above, the purge gas is introduced into only the propagation space as an optical path of the measurement light. As the propagation space is a space having a small volume, the consumption of the purge gas can be reduced. In addition, the explosion-proof gas different from the purge gas is introduced into the internal space of the housing having a large volume. The explosion-proof gas is inexpensive and suitable for explosion-proof measures, and hence it is possible to take explosion-proof measures for the internal space of the housing inexpensively and appropriately. In addition, by introducing the explosion-proof gas into the internal space of the housing, it is possible to cool members housed in the internal space of the housing. Furthermore, the measurement light passes through the propagation space filled with the purge gas and is guided to the filling unit filled with the sample gas. In this way, it is possible to prevent the measurement light from being absorbed in the propagation space before the measurement light reaches the filling unit. As a result, the gas to be measured can be accurately analyzed using the measurement light.

### Advantageous Effects

In the analysis device for a gas to be measured having a housing, it is possible to appropriately take explosion-proof measures for an analysis device, while suppressing consumption of a purge gas filling the optical path of the measurement light.

### BRIEF DESCRIPTION OF DRAWINGS

FIG. 1 is a diagram illustrating a structure of an analysis device.

FIG. 2 is a diagram illustrating a structure of a jig.

FIG. 3 is a diagram schematically illustrating a state where the jig is attached to a propagation unit.

FIG. 4 is a diagram illustrating a gas flow structure of the analysis device.

FIG. 5 is a diagram illustrating another example of the gas flow structure of the analysis device.

### DESCRIPTION OF EMBODIMENTS

### 1. First Embodiment

### (1) Structure of Analysis Device

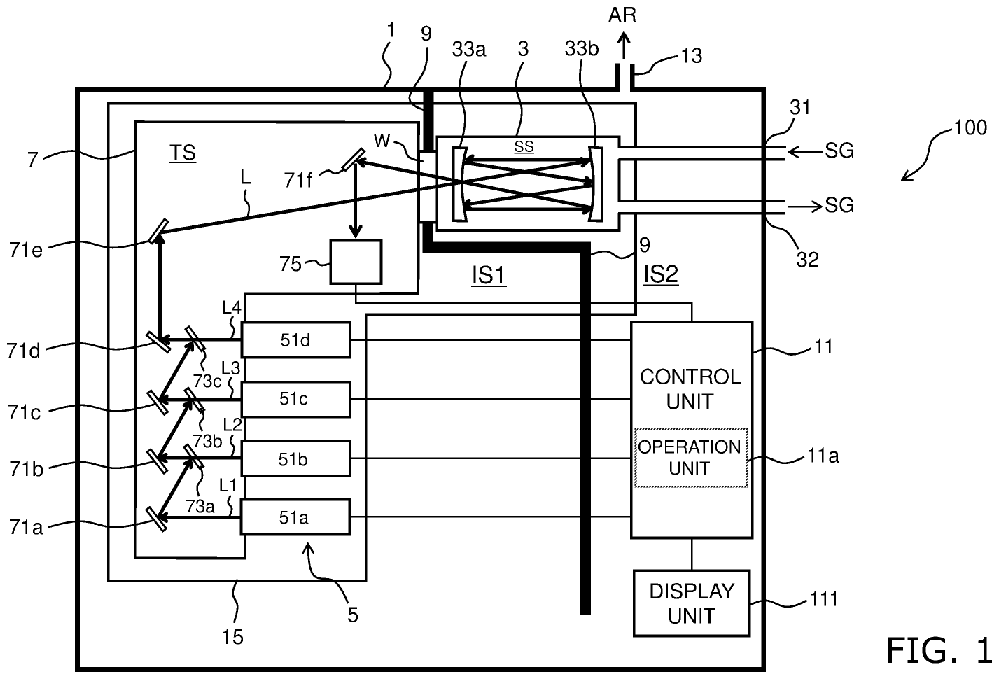

Hereinafter, an analysis device 100 is described. First, with reference to FIG. 1, a structure of the analysis device 100, except a structure that introduces a purge gas PG and an explosion-proof gas EP, is described. FIG. 1 is a diagram illustrating a structure of the analysis device except for a gas introduction unit. The analysis device 100 is a device that is configured to analyze a gas to be measured contained in a sample gas SG such as an exhaust gas discharged from a flue, for example.

The gas to be measured that can be measured by the analysis device 100 is, for example, carbon dioxide (CO₂), carbon monoxide (CO), sulphur oxide (SOx) (such as sulfur dioxide (SO₂)), ammonia (NH₃), nitrogen oxide (NOx) (such as nitrogen monoxide (NO), nitrogen dioxide (NO₂), or nitrous oxide (N₂O)), hydrogen chloride (HCl), water (H₂O), various hydrocarbons (such as methane (CH₄), ethane (C₂H₆), acetylene (C₂H₂), propane (C₃H₈), ethylene (C₂H₄), hexane (n-C₆H₁₄), propylene (C₃H₆), isobutene (i-C₄H₈), butane (n-C₄H₁₀), propane (C₃H₈), cyclohexane (C₆H₁₂), butadiene (C₄H₆), isobutane (i-C₄H₁₀), isopentane (i-C₅H₁₂), or toluene (C₆H₅CH₃)), hydrogen sulfide (H₂S), methanol (CH₃OH), phosgene (COCl₂), chloroethylene (C₂H₃Cl), methyl nitrite (CH₃ONO), hydrogen (H₂), hydrogen fluoride (HF), trifluoropropene (C₃H₃F₃), or the like. The gas to be measured is not limited to a single gas, but may be a gas mixture containing a plurality of gases described above.

The analysis device 100 has a structure in which components for analyzing the gas to be measured are isolated from the external space, and the internal space containing components of the analysis device 100 is filled with a gas that does not contain combustible gases. This structure is referred to as an “explosion-proof structure”. In addition, in the following description, the gas filled in the internal space is referred to as an “explosion-proof gas”.

As the analysis device 100 has the explosion-proof structure, even if the ambient environment outside the analysis device 100 contains a combustible gas, the gas to be measured can be safely analyzed. As illustrated in FIG. 1, the analysis device 100 includes a housing 1, a filling unit 3, an irradiation unit 5, a propagation unit 7, a partition plate 9, and a control unit 11.

The housing 1 constitutes a main body of the analysis device 100, and houses the filling unit 3, the irradiation unit 5, and the propagation unit 7 in its internal space. The housing 1 is structured as an internal pressure explosion-proof container. Specifically, the internal space of the housing 1 is filled with the explosion-proof gas EP at a pressure higher than that of the outside of the housing 1. As the housing 1 is constituted as the internal pressure explosion-proof container, the internal space of the housing 1 can be safely protected from explosion. The housing 1 is provided with an exhaust port 13 near the position of the filling unit 3. The explosion-proof gas EP introduced into the internal space of the housing 1 is discharged from the exhaust port 13.

The filling unit 3 is a member having a sampling space SS. The sampling space SS of the filling unit 3 can be filled with the sample gas SG. In order to fill the sample gas SG into the sampling space SS, the filling unit 3 is provided with an inlet 31 that is configured to introduce the sample gas SG into the sampling space SS and an outlet 32 that is configured to discharge the sample gas SG in the sampling space SS. During analysis of the gas to be measured contained in the sample gas SG, the sample gas SG is filled into the sampling space SS through the inlet 31 and then is discharged from the outlet 32, and this flow is continued.

The sampling space SS is at a pressure lower than that of the internal space of the housing 1 and a propagation space TS of the propagation unit 7, also when the sample gas SG is filled therein. In this way, the sample gas SG filled in the sampling space SS is prevented from leaking to the internal space of the housing 1 or the propagation space TS of the propagation unit 7, and explosion-proof measures for the analysis device 100 can be taken appropriately.

In addition, the sampling space SS is provided with a first reflection member 33a and a second reflection member 33b. The first reflection member 33a and the second reflection member 33b allow measurement light L entering the sampling space SS to be multiply reflected and then propagated to the propagation space TS of the propagation unit 7 (described later). In this way, the length of an optical path of the measurement light L that passes through the sample gas SG filled in the sampling space SS can be increased.

The first reflection member 33a is disposed near the propagation unit 7 in the sampling space SS. The first reflection member 33a reflects the measurement light L, and propagates the measurement light L to the second reflection member 33b. Further, the multiply reflected measurement light L propagates to the propagation unit 7. Therefore, the first reflection member 33a is, for example, a member that can reflect the measurement light L (such as a mirror), and has a passing hole for the measurement light L at a predetermined position. In this first reflection member 33a, the measurement light L that has come from the propagation unit 7 passes through the passing hole to enter the sampling space SS. The measurement light L that has entered the sampling space SS is multiply reflected between the first reflection member 33a and the second reflection member 33b, and then returns to the propagation unit 7 through the passing hole of the first reflection member 33a.

The second reflection member 33b is disposed at a position apart from the propagation unit 7 in the sampling space SS. The second reflection member 33b reflects the measurement light L to the first reflection member 33a. The second reflection member 33b is, for example, a member such as a mirror that reflects the measurement light L.

The irradiation unit 5 emits the measurement light L. The measurement light L emitted from the irradiation unit 5 is guided by the propagation unit 7 to the filling unit 3. The irradiation unit 5 comprises a plurality of light sources 51a to 51d. The plurality of light sources 51a to 51d emit a plurality of element light beams L1 to L4 having different wavelength bands, respectively. The plurality of light sources 51a to 51d are each a laser oscillator such as a semiconductor laser device, for example.

The element light beams L1 to L4 emitted from the plurality of light sources 51a to 51d are multiplexed in the propagation space TS of the propagation unit 7 to be the measurement light L, which propagates to the filling unit 3. In other words, the measurement light L includes the plurality of element light beams L1 to L4 having different wavelength regions. As the measurement light L is includes the element light beams L1 to L4, it is possible to measure the gas to be measured of a plurality of types having absorption peaks in the wavelength bands of the element light beams L1 to L4, respectively, for example.

In addition, as the measurement light L includes the plurality of element light beams L1 to L4, it is also possible to measure an influence of an interfering gas component with respect to the gas to be measured, for example. The interfering gas component means a component that has its absorption peak at the same position as or a similar position to a part of absorption peaks of the gas to be measured, and hence affects analysis results of the gas to be measured. If the influence of the interfering gas component can be measured, the gas to be measured can be accurately analyzed by removing the influence of the interfering gas component from a measurement result of the measurement light L received by a detector 75 (described later). Note that “removing” described above means not only completely removing the influence of the interfering gas component but also reducing the influence to be lower than that before the removing operation.

The propagation unit 7 is disposed between the filling unit 3 and the irradiation unit 5. Specifically, as illustrated in FIG. 1, the propagation unit 7 has an L-shape, and the plurality of light sources 51a to 51d are each inserted partially and fixed to a portion thereof corresponding to one side of the L-shape. On the other hand, the filling unit 3 is fixed to an end portion of the other side of the L-shape of the propagation unit 7 via an optical window W.

The propagation unit 7 is constituted as the internal pressure explosion-proof container, and has the propagation space TS. The propagation space TS of the propagation unit 7 is made of a solid metal block that is hollowed to form a space, and a lid member that closes the space formed in the solid metal block. The metal members (the solid metal block and the lid member) constituting the propagation unit 7 is made of aluminum, for example. When making the propagation unit 7 of aluminum, a black alumite treatment is performed on it to have a black color. In this way, a robust propagation unit 7 can be easily formed. As described above, by forming the propagation unit 7 as the internal pressure explosion-proof container, explosion-proof can be taken safely for the propagation space TS as the internal space of the propagation unit 7.

A plurality of mirrors are arranged in the propagation space TS of the propagation unit 7. Propagation paths of the element light beams L1 to L4 emitted from the irradiation unit 5 are changed by these mirrors, and they propagate to the filling unit 3. In other words, the measurement light L is reflected by at least one mirror and propagates in the propagation space TS. Specifically, a first mirror 71a, a second mirror 71b, a third mirror 71c, a fourth mirror 71d, and a fifth mirror 71e are arranged in the propagation space TS. In addition, a first optical element 73a, a second optical element 73b, and a third optical element 73c are arranged in the propagation space TS.

The first mirror 71a reflects the element light beam L1 to the first optical element 73a. The first optical element 73a reflects the element light beam L1 and transmits the element light beam L2. In other words, the first optical element 73a multiplexes the element light beam L1 and the element light beam L2. The element light beam L1 and the element light beam L2 multiplexed by the first optical element 73a propagate on the same optical path to the second mirror 71b.

The second mirror 71b reflects the element light beam L1 and the element light beam L2 multiplexed by the first optical element 73a to the second optical element 73b. The second optical element 73b reflects the multiplexed element light beams L1 and L2, and transmits the element light beam L3. In other words, the second optical element 73b multiplexes the element light beam L1, the element light beam L2, and the element light beam L3. The element light beams L1 to L3 multiplexed by the second optical element 73b propagate on the same optical path to the third mirror 71c.

The third mirror 71c reflects the element light beams L1 to L3 multiplexed by the second optical element 73b to the third optical element 73c. The third optical element 73c reflects the multiplexed element light beams L1 to L3, and transmits the element light beam L4. In other words, the third optical element 73c multiplexes the element light beam L1, the element light beam L2, the element light beam L3, and the element light beam L4. The element light beams L1 to L4 multiplexed by the third optical element 73c propagate on the same optical path to the fourth mirror 71d.

The fourth mirror 71d reflects the element light beams L1 to L4 multiplexed by the third optical element 73c to the fifth mirror 71e. In this way, the multiplexed element light beams L1 to L4 become the measurement light L.

The fifth mirror 71e changes the propagation path of the measurement light L, in which the element light beams L1 to L4 are multiplexed, to the direction to the filling unit 3. The measurement light L whose propagation path has been changed by the fifth mirror 71e passes through the optical window W and propagates in the sampling space SS of the filling unit 3. These mirrors are disposed in the propagation space TS, and hence it is possible to keep clean state of the mirror by preventing unnecessary gas from entering externally.

In addition, the detector 75 is disposed in the propagation space TS of the propagation unit 7. The detector 75 detects the measurement light L that has entered the sampling space SS and has been multiply reflected. The detector 75 is a quantum type photoelectric device, for example. Other than that, a semiconductor detection device, a thermal type photodetection device such as a thermopile, or the like can be used as the detector 75. As illustrated in FIG. 1, the propagation space TS is provided with a sixth mirror 71f, which changes the propagation path of the measurement light L, which has been multiply reflected in the sampling space SS and has returned to the propagation unit 7, to the direction to the detector 75.

As described later, the propagation space TS is purged using the purge gas PG. In this way, the purge gas PG flows in the propagation space TS, so as to keep clean state of the first to sixth mirrors 71a to 71f, and the detector 75 disposed in the propagation space TS.

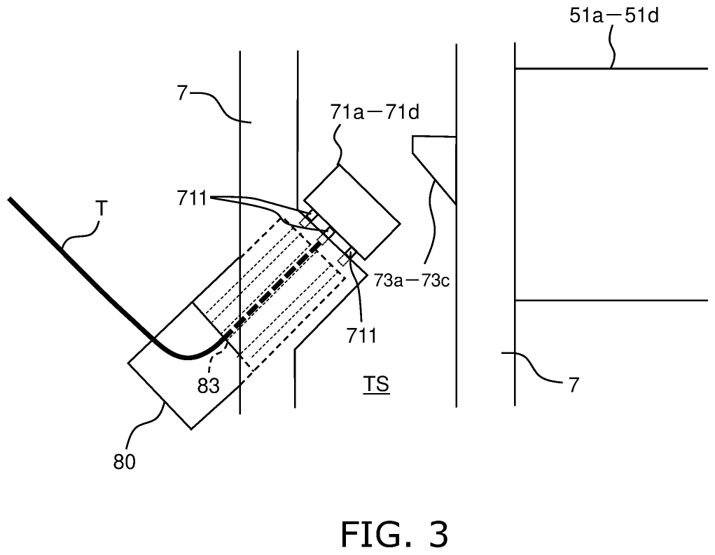

The optical path of the measurement light L from the propagation unit 7 to the filling unit 3 is determined by the first to sixth mirrors 71a to 71f. In order to appropriately propagate the measurement light L to the filling unit 3, the inclination angles of these mirrors are adjustable. For instance, by rotating a crew 711 (FIG. 3) disposed in the propagation space TS, the first to fourth mirrors 71a to 71d can adjust their inclination angles, respectively.

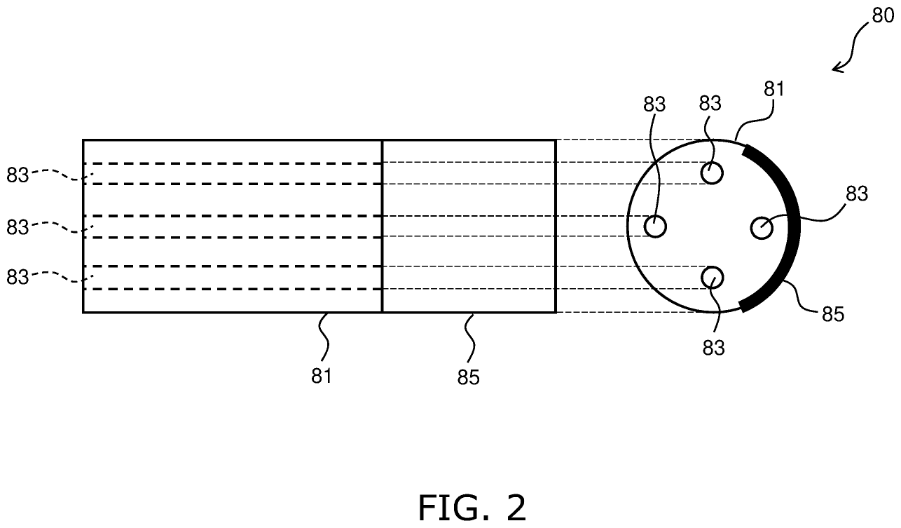

In order to operate the screw 711 described above, in this embodiment, a jig 80 is used, which allows a tool T that operates the screw 711 to reach the screw 711 in the propagation space TS. As illustrated in FIG. 2, the jig 80 has a main body 81, through holes 83, and a handgrip 85. FIG. 2 is a diagram illustrating a structure of the jig.

The through hole 83 is a hole formed to penetrate the main body 81. The through hole 83 is formed at a position facing the screw 711 provided to the mirror when the main body 81 is attached to the propagation unit 7. The jig 80 illustrated in FIG. 2 has four through holes 83. The handgrip 85 is grasped by a user when the jig 80 is attached to the propagation unit 7.

As illustrated in FIG. 3, the jig 80 is attached to the propagation unit 7 by fitting the main body 81 into a hole formed in the propagation unit 7. When the jig 80 is attached to the propagation unit 7, the through holes 83 face the screws 711 provided to the first to fourth mirrors 71a to 71d, respectively. Therefore, by inserting the tool T into one of the through holes 83, the tip of the tool T can reach the screw 711. In this way, by using the jig 80 described above, even if the first to fourth mirrors 71a to 71d disposed in the propagation space TS cannot be visually seen, the tool T that adjusts the mirror can reach an appropriate position. FIG. 3 is a diagram schematically illustrating a state where the jig is attached to the propagation unit.

The analysis device 100 with reference to FIG. 1 is described again below. The partition plate 9 divides the internal space of the housing 1 into a first internal space IS1 and a second internal space IS2. As illustrated in FIG. 1, the irradiation unit 5 and the propagation unit 7 exist in the first internal space IS1. On the other hand, the filling unit 3 exists in the second internal space IS2.

The filling unit 3 in which the sample gas SG is filled becomes high temperature. In contrast, it is preferred that the temperatures of the irradiation unit 5 and the propagation unit 7 change as little as possible from room temperature (temperature when the irradiation unit 5 and the propagation unit 7 are attached to the housing 1). It is because the optical axis of the measurement light L might deviate from the appropriately adjusted state if the temperature of the irradiation unit 5 or the propagation unit 7 varies. Therefore, by disposing the partition plate 9 in the internal space of the housing 1 that houses the filling unit 3, the irradiation unit 5, and the propagation unit 7, it is possible to spatially separate the irradiation unit 5 and the propagation unit 7 from the filling unit 3 that becomes high temperature, so that temperature variations of the irradiation unit 5 and the propagation unit 7 can be reduced. As a result, it is possible to reduce deviation of the optical axis of the measurement light L from the appropriately adjusted state.

As illustrated in FIG. 1, the filling unit 3, the irradiation unit 5, and the propagation unit 7 are fixed not directly to the housing 1 but to one fixing plate 15, and hence they are fixed to the housing 1 via the fixing plate 15.

The relative positions and orientations of the filling unit 3, the irradiation unit 5, and the propagation unit 7 are appropriately adjusted, so that the measurement light L emitted from the irradiation unit 5 can reach the filling unit 3 via the propagation unit 7. If the filling unit 3, the irradiation unit 5, and the propagation unit 7 are directly fixed to the housing 1, the relative positions and orientations may be varied due to deformation or the like of the housing 1. Therefore, in this embodiment, the filling unit 3, the irradiation unit 5, and the propagation unit 7 are fixed to the same fixing plate 15, and it is possible to reduce a variation of the relative positions and orientations of the filling unit 3, the irradiation unit 5, and the propagation unit 7, due to an influence of deformation or the like of the housing 1. As a result, the measurement light L emitted from the irradiation unit 5 can reach the filling unit 3 via the propagation unit 7 (propagation space TS) without affected by a deformation or the like of the housing 1.

The control unit 11 is a computer system constituted of a CPU, a storage device (such as a RAM and a ROM), and various interfaces. The control unit 11 may be a system equipped with the above devices discretely, or may be a system on chip (SoC) constituted of a single chip in which the above devices are integrated. The control unit 11 controls components of the analysis device 100. In addition, the control unit 11 includes an operation unit 11a and performs calibration of the analysis device 100 and analysis of the gas to be measured on the basis of intensity of the measurement light L detected by the detector 75.

A part or the whole of controls and information processing performed by the control unit 11 may be realized by executing a program stored in the storage device of the computer system constituting the control unit 11. In addition, a part of the controls and information processing may be realized by hardware.

The control unit 11 is connected to a display unit 111 that displays information and a display screen about the analysis device 100, such as the analysis result of the gas to be measured by the analysis device 100. The display unit 111 is a flat display such as a liquid crystal display or an organic EL display, for example. In addition, the display unit 111 may include information input means such as a touch panel.

### (2) Gas Flow Structure of Analysis Device

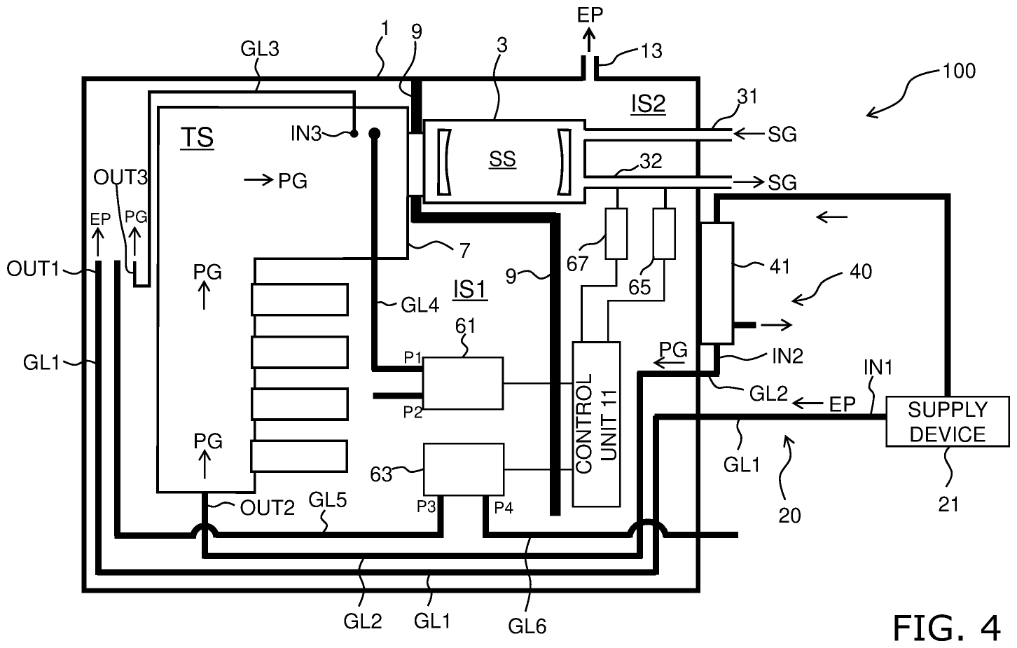

Next, with reference to FIG. 4, a gas flow structure of the analysis device 100 is described. FIG. 4 is a diagram illustrating the gas flow structure of the analysis device. In order to make the analysis device 100 have the explosion-proof structure, the internal space of the housing 1 that houses the filling unit 3, the irradiation unit 5, and the propagation unit 7 is filled with the explosion-proof gas EP without combustible gases, at a first pressure higher than the pressure outside the housing 1. In addition, the propagation space TS in which the measurement light L propagates is filled with the purge gas PG without the gas to be measured, at a second pressure higher than the pressure of the internal space of the housing 1, in order to prevent the measurement light L from being absorbed before reaching the filling unit 3.

Therefore, the analysis device 100 includes an explosion-proof gas introduction unit 20 that is configured to introduce the explosion-proof gas EP into the internal space of the housing 1, and a purge gas introduction unit 40 that is configured to introduce the purge gas PG into the propagation space TS.

The explosion-proof gas introduction unit 20 is equipped with a first gas line GL1 and a supply device 21. An inlet IN1 of the first gas line is connected to the supply device 21 in the outside. On the other hand, an outlet OUT1 of the first gas line GL1 is disposed in an upper part of the first internal space IS1 of the housing 1 and in a vicinity of the propagation unit 7. The supply device 21 is a device that supplies the explosion-proof gas EP to the first gas line GL1. The supply device 21 is a device that is configured to generate instrument air, for example, which includes a compressor to compress the air, and various filters to remove dust, oil, and the like contained in the air. In other words, in this embodiment, the explosion-proof gas EP is the instrument air that does not contain combustible gases. Note that when the analysis device 100 operates, the explosion-proof gas EP is constantly supplied from the supply device 21.

The explosion-proof gas introduction unit 20 having the structure described above constantly introduces the explosion-proof gas EP from the upper part of the first internal space IS1 and the vicinity of the propagation unit 7. As the partition plate 9 exists, the explosion-proof gas EP introduced into the upper part of the first internal space IS1 and the vicinity of the propagation unit 7 flows from the upper part to the lower part of the first internal space IS1, and enters the second internal space IS2 from the lower part of the first internal space IS1. The gas that enters the second internal space IS2 flows from the lower part to the upper part of the second internal space IS2, and is discharged from the exhaust port 13.

As the explosion-proof gas EP is constantly supplied when the analysis device 100 operates, the explosion-proof gas EP is constantly supplied to the internal space of the housing 1 when the analysis device 100 operates, and is discharged from the exhaust port 13 by the flow described above. In this way, the internal space of the housing 1 is constantly filled with the flesh explosion-proof gas EP at the first pressure when the analysis device 100 operates.

In addition, as the explosion-proof gas EP is first introduced to the first internal space IS1, the unheated explosion-proof gas EP can first reach the irradiation unit 5 and the propagation unit 7. Furthermore, as the partition plate 9 exists, the explosion-proof gas EP can be prevented from being heated by thermal conduction from the filling unit 3 that becomes high temperature. As a result, the irradiation unit 5 and the propagation unit 7 (particularly the detector 75), which need to be cooled, can be efficiently cooled.

The purge gas introduction unit 40 includes a second gas line GL2 and a separating unit 41. An inlet IN2 of the second gas line GL2 is connected to the separating unit 41. On the other hand, an outlet OUT2 of the second gas line GL2 is connected to the lower part of the propagation unit 7. The upper part of the propagation unit 7 is connected to an inlet IN3 of a third gas line GL3. An outlet OUT3 of the third gas line GL3 is disposed in a vicinity of the outlet OUT1 of the first gas line GL1.

The separating unit 41 separate the purge gas PG from a gas. In this embodiment, the separating unit 41 separates the purge gas PG from the instrument air supplied from the supply device 21.

The separating unit 41 is a member called a “N2 separator”, which has a hollow member in which polyimide hollow fiber membranes or gas permeation membranes are filled, for example. The N2 separator introduces compressed air to the hollow fiber membranes or the gas permeation membrane, so that the air is separated into a nitrogen rich gas obtained by removing components other than nitrogen (including the gas to be measured), and a remnant gas as the remaining gas when removing nitrogen from the air. The separating unit 41 as the N2 separator discharges the nitrogen-rich gas as the purge gas PG to the second gas line GL2. On the other hand, the separating unit 41 discharges the remnant gas to the outside.

The hollow fiber membrane described above is made of, for example, polyimide, polyamide, polysulfone, cellulose acetate and its derivative, polyphenylene oxide, polysiloxane, basically microporous polymer, mixed matrix membrane, facilitated transport membrane, polyethylene oxide, polypropylene oxide, carbon membrane, zeolite, or a mixture of them.

As illustrated in FIG. 4, the separating unit 41 is disposed outside the housing 1. In this way, the separating unit 41 can discharge the remnant gas to the outside of the housing 1. As a result, it is possible to prevent the remnant gas containing much oxygen from being introduced to the internal space of the housing 1, so that explosion-proof of the internal space of the housing 1 can be taken appropriately.

Note that, instead of generating the purge gas PG by the separating unit 41 described above, it may be possible to connect a gas cylinder, which supplies the purge gas PG, to the inlet IN2 of the second gas line GL2. In other words, it may be possible to supply the purge gas PG directly from the gas cylinder. As the gas cylinder that supplies the purge gas PG, for example, a gas cylinder such as a nitrogen cylinder that supplies an inert gas can be used.

In the same manner as the explosion-proof gas EP, the purge gas PG is constantly supplied to the second gas line GL2 when the analysis device 100 operates.

The purge gas introduction unit 40 having the structure described above introduces the purge gas PG from the lower part of the propagation space TS. The purge gas PG introduced from the lower part of the propagation space TS flows to the upper part of the propagation space TS, and is discharged to the internal space of the housing 1 via the third gas line GL3. In addition, similarly to the explosion-proof gas EP, the purge gas PG is constantly supplied when the analysis device 100 operates. Therefore, the purge gas PG is constantly supplied to the propagation space TS when the analysis device 100 operates, and is discharged to the internal space of the housing 1 by the flow described above. In this way, when the analysis device 100 operates, the propagation space TS is constantly filled with the fresh purge gas PG at the second pressure higher than the pressure of the internal space of the housing 1 (the first pressure).

As illustrated in FIG. 4, the third gas line GL3 has a diameter smaller than that of other gas lines. By setting the small diameter of the third gas line GL3 for discharging the purge gas PG, the pressure of the purge gas PG in the propagation space TS can be easily increased.

The analysis device 100 includes a first differential pressure gauge 61 and a second differential pressure gauge 63. The first differential pressure gauge 61 and the second differential pressure gauge 63 are each a device having two ports, to measure a pressure difference between one port and the other port.

One port P1 out of the two ports of the first differential pressure gauge 61 is connected to a vicinity of the third gas line GL3 in the propagation space TS via a fourth gas line GL4, and the other port P2 is connected to the internal space of the housing 1. In other words, the first differential pressure gauge 61 measures a difference between the pressure adjacent to the outlet of the purge gas PG in the propagation space TS and the pressure in the internal space of the housing 1.

As a result of consideration, the inventor found that, in the propagation space TS filled with the purge gas PG, a vicinity of the outlet of the purge gas PG in the propagation space TS has the lowest pressure. Based on this knowledge, a pressure difference between the lowest pressure in the propagation space TS and the pressure in the internal space of the housing 1 is measured with the first differential pressure gauge 61. In this way, the first differential pressure gauge 61 can securely detect whether or not the pressure in the propagation space TS is higher than the pressure in the internal space of the housing 1, i.e., whether or not there is a possibility that the explosion-proof gas EP can enter the propagation space TS. It is because if the lowest pressure in the propagation space TS is higher than the pressure in the internal space of the housing 1, the pressure at any other place in the propagation space TS is surely higher than the pressure in the internal space of the housing 1.

One port P3 out of two ports of the second differential pressure gauge 63 is connected to a vicinity of the outlet OUT1 of the first gas line GL1 in the internal space of the housing 1 via a fifth gas line GL5. The other port P4 is connected to the outside space of the housing 1 via a sixth gas line GL6. In other words, the second differential pressure gauge 63 measures a difference between the pressure adjacent to the exhaust port of the explosion-proof gas EP in the internal space of the housing 1 and the pressure outside the housing 1.

As a result of consideration, the inventor found that, in the internal space of the housing 1 filled with the explosion-proof gas EP, a vicinity of the exhaust port that introduces the explosion-proof gas EP into the internal space of the housing 1 (the outlet OUT1 of the first gas line GL1) has the lowest pressure. Based on this knowledge, a pressure difference between the lowest pressure in the internal space of the housing 1 and the pressure outside the housing 1 is measured with the second differential pressure gauge 63. In this way, the second differential pressure gauge 63 can securely detect whether or not the pressure in the internal space of the housing 1 is higher than the pressure outside the housing, i.e., whether or not the external gas enters the internal space of the housing 1 so that the explosion-proof measures can be inappropriate. It is because if the lowest pressure in the internal space of the housing 1 is higher than the pressure outside the housing 1, the pressure at any other place in the internal space of the housing 1 is surely higher than the pressure outside the housing 1.

In addition, if the second differential pressure gauge 63 indicates that the pressure in the internal space of the housing 1 is higher than the pressure outside the housing 1, and if the first differential pressure gauge 61 indicates that the pressure in the propagation space TS is higher than the pressure in the internal space of the housing 1, the pressure in the propagation space TS is surely higher than the pressure outside the housing 1. In other words, it can be secured that the gas outside the housing 1 cannot enter the propagation space TS.

Based on a detection result whether or not the pressure in the propagation space TS is higher than the pressure in the internal space of the housing 1, the first differential pressure gauge 61 performs control concerning supply of the purge gas PG. In addition, on the basis of a detection result whether or not the pressure in the internal space of the housing 1 is higher than the pressure outside the housing, the second differential pressure gauge 63 performs control concerning supply of the explosion-proof gas EP.

The analysis device 100 includes a pressure switch 65. The pressure switch 65 is connected to the outlet 32 of the sample gas SG of the filling unit 3. The pressure switch 65 detects whether or not the pressure of the sampling space SS of the filling unit 3 has become equal to or more than a predetermined pressure lower than the pressure in the internal space of the housing 1. The pressure switch 65 may be a pressure switch that is in off state if the pressure in the sampling space SS is lower than the predetermined pressure described above, while it becomes on state if the pressure of the sampling space SS becomes the predetermined pressure or higher, or may be a pressure switch having on and off states opposite to the above.

In addition, based on the state of the pressure switch 65, and the pressure difference between the lowest pressure in the propagation space TS and the pressure in the internal space of the housing 1, measured by the first differential pressure gauge 61, it is possible to monitor whether or not the pressure in the propagation space TS of the propagation unit 7 is higher than the pressure in the sampling space SS. Specifically, for example, if the pressure switch 65 detects that the pressure in the sampling space SS is lower than the predetermined pressure described above (e.g., the pressure switch 65 is in off state), and if the first differential pressure gauge 61 detects that the lowest pressure in the propagation space TS is higher than the pressure in the internal space of the housing 1, it can be determined that the pressure in the propagation space TS is higher than the pressure in the sampling space SS.

As described above, in the analysis device 100, four types of magnitude relationships between pressures, which include the magnitude relationship between the pressure in the internal space of the housing 1 and the pressure outside the housing 1, the magnitude relationship between the pressure in the propagation space TS and the pressure in the internal space of the housing 1, the magnitude relationship between the pressure in the propagation space TS and the pressure in the sampling space SS, and the magnitude relationship between the pressure in the internal space of the housing 1 and the pressure in the sampling space SS, are measured by three devices, which include the first differential pressure gauge 61, the second differential pressure gauge 63, and the pressure switch 65. In this way, the analysis device 100 efficiently measures the magnitude relationships among pressures at many places with a small number of devices.

The analysis device 100 includes a pressure gage 67. The pressure gage 67 is connected to the outlet 32 of the sample gas SG of the filling unit 3, so as to measure the pressure in the sampling space SS. The pressure in the sampling space SS is used for correcting the analysis result of the gas to be measured contained in the sample gas SG filled in the sampling space SS.

### (3) Operation of Analysis Device

Hereinafter, an operation of the analysis device 100 is described. When activating the analysis device 100, introduction of the purge gas PG into the propagation space TS of the propagation unit 7 is started first. After starting the introduction of the purge gas PG into the propagation space TS, the first differential pressure gauge 61 determines whether or not the pressure in the propagation space TS has become the second pressure, based on the pressure difference between the lowest pressure in the propagation space TS and the pressure in the internal space of the housing 1. The first differential pressure gauge 61 continues supply of the purge gas PG until a first time period elapses after the pressure in the propagation space TS becomes the second pressure. The flow rate of the purge gas PG introduced into the propagation space TS and the first time period described above can be appropriately set to optimal values based on the volume or the like of the propagation space TS.

After the supply of the purge gas PG is continued for the first time period after the pressure in the propagation space TS becomes the second pressure, introduction of the explosion-proof gas EP into the internal space of the housing 1 is started while maintaining the supply of the purge gas PG. After starting the introduction of the explosion-proof gas EP into the internal space of the housing 1, the second differential pressure gauge 63 determines whether or not the pressure in the internal space of the housing 1 has become the first pressure, based on the pressure difference between the lowest pressure in the internal space of the housing 1 and the pressure outside the housing 1. The second differential pressure gauge 63 continues the supply of the explosion-proof gas EP until a second time period elapses after the pressure in the internal space of the housing 1 becomes the first pressure. The flow rate of the explosion-proof gas EP introduced into the internal space of the housing 1, and the second time period described above can be appropriately set to optimal values based on the volume or the like of the internal space of the housing 1.

As described above, the propagation space TS of the propagation unit 7 is filled with the purge gas PG, and the measurement light L can be prevented from being absorbed during propagation in the propagation space TS and being decreased in its intensity. In addition, as the internal space of the housing 1 is filled with the explosion-proof gas EP while the propagation space TS of the propagation unit 7 is filled with the purge gas PG, explosion-proof measures for the analysis device 100 can be taken appropriately. In other words, the purge gas PG also works as the explosion-proof gas EP.

While maintaining the supply of the purge gas PG, the supply of the explosion-proof gas EP is continued for the second time period after the pressure in the internal space of the housing 1 becomes the first pressure. After that, if it is determined that the pressure in the sampling space SS is lower than a predetermined pressure lower than the pressure of the internal space of the housing 1, the control unit 11 starts power supply to the light sources 51a to 51d of the irradiation unit 5 and the detector 75 disposed in the propagation space TS, while maintaining the supply of the purge gas PG and the explosion-proof gas EP. In this way, the analysis device 100 can analyze the gas to be measured.

When the analysis device 100 is enabled to analyze the gas to be measured, the sample gas SG is supplied to the sampling space SS of the filling unit 3 to fill the sampling space SS with the sample gas SG. While maintaining the supply of the supply of the sample gas SG to the sampling space SS, the control unit 11 controls the light sources 51a to 51d so as to output the measurement light L to the sampling space SS of the filling unit 3. The operation unit 11a receives from the detector 75 a detection signal of the measurement light L after passing through the sampling space SS, and calculates intensities in the wavelength regions of the element light beams L1 to L4 of the detected measurement light L, on the basis of the detection signal.

After that, the operation unit 11a calculates the concentration of the gas to be measured in the sample gas SG, based on a ratio or the like between the intensity of the measurement light L before passing through the sampling space SS filled with the sample gas SG and the intensity of the measurement light L after passing through the sampling space SS filled with the sample gas SG.

For instance, if the gas to be measured can absorb all the element light beams L1 to L4, the operation unit 11a calculates the concentration of an interference gas that interferes with the analysis result of the gas to be measured, based on intensities of the element light beams L1 to L4, and can accurately calculate the concentration of the gas to be measured in consideration of an influence of this interference gas.

Other than that, if there are plurality of gases to be measured, the operation unit 11a can calculate concentration of one of the gases to be measured based on intensity of one of the element light beams L1 to L4 that the gas to be measured can absorb, among the element light beams L1 to L4, for example.

In the analysis device 100 described above, the purge gas PG is introduced into only the propagation space TS as the optical path of the measurement light L. As illustrated in FIG. 1 and others, the propagation space TS is a space having a small volume compared with the internal space of the housing 1, and hence the consumption of the purge gas PG to be introduced to the optical path of the measurement light L can be reduced.

On the other hand, the explosion-proof gas EP different from the purge gas PG is introduced into the internal space of the housing 1 having a large volume. The explosion-proof gas EP is inexpensive and suitable for explosion-proof measures, and hence the explosion-proof measures for the internal space of the housing 1 can be taken inexpensively and appropriately. In addition, by introducing the explosion-proof gas EP into the internal space of the housing 1, the members housed in the internal space of the housing 1 can be cooled.

In addition, by disposing the partition plate 9 in the internal space of the housing 1, the internal space of the housing 1 is divided into the first internal space IS1 in which the irradiation unit 5 and the propagation unit 7 exist, and the second internal space IS2 in which the filling unit exists, and hence the irradiation unit 5 and the propagation unit 7 can be spatially separated from the filling unit 3 that becomes high temperature. As a result, the explosion-proof gas EP existing in the first internal space IS1 is prevented from being heated by the filling unit 3 at a high temperature, and thus the irradiation unit 5 and the propagation unit 7 can be efficiently cooled.

Furthermore, the measurement light L to be used for analyzing the gas to be measured passes through the propagation space TS filled with the purge gas PG, and is guided to the filling unit 3 filled with the sample gas SG. In this way, the measurement light L can be prevented from being absorbed in the propagation space TS before the measurement light L reaches the filling unit 3. As a result, using the measurement light L, the gas to be measured contained in the sample gas SG can be accurately analyzed.

### 2. Other Embodiments

Although a plurality of embodiments of the present invention are described above, the present invention is not limited to the embodiments described above, but can be variously modified within the scope of the invention without deviating from the spirit thereof. In particular, a plurality of embodiments and variations described in this specification can be arbitrarily modified as necessary. (A) In the propagation space TS of the analysis device 100, the first mirror 71a, the second mirror 71b, the third mirror 71c, the fourth mirror 71d, the fifth mirror 71e, and the sixth mirror 71f are arranged as described above. The arrangement of these mirrors may be modified according to a positional relationship between the filling unit 3 and the light sources 51a to 51d in the housing 1.

For instance, if the light sources 51a to 51d are disposed close to the filling unit 3, and hence a distance for the measurement light L to propagate from the light sources 51a to 51d to the filling unit 3 in the propagation space TS is decreased, the number of mirrors disposed in the propagation space TS may be decreased, or no mirror may be disposed in the propagation space TS.

(B) In the analysis device 100, if the purge gas PG is generated by the separating unit 41, it may be possible to determine deterioration of the separating unit 41. This determination is performed as follows, for example. In the state where the propagation space TS and/or the sampling space SS is filled with the purge gas PG generated by the separating unit 41, the measurement light L is output, the intensity of the measurement light L after passing through the propagation space TS and/or the sampling space SS is measured by the detector 75, and based on how much the measured intensity of the measurement light L has decreased from the original intensity, the deterioration degree of the separating unit 41 can be determined.

(C) For instance, if a gas such as the instrument air without components inappropriate for explosion-proof is supplied from the supply device 21, it may be possible to dispose the separating unit 41 in the housing 1 and to use a remnant gas other than the purge gas PG as the explosion-proof gas EP, among components generated by the gas separation of the gas supplied from the supply device 21 by the separating unit 41.

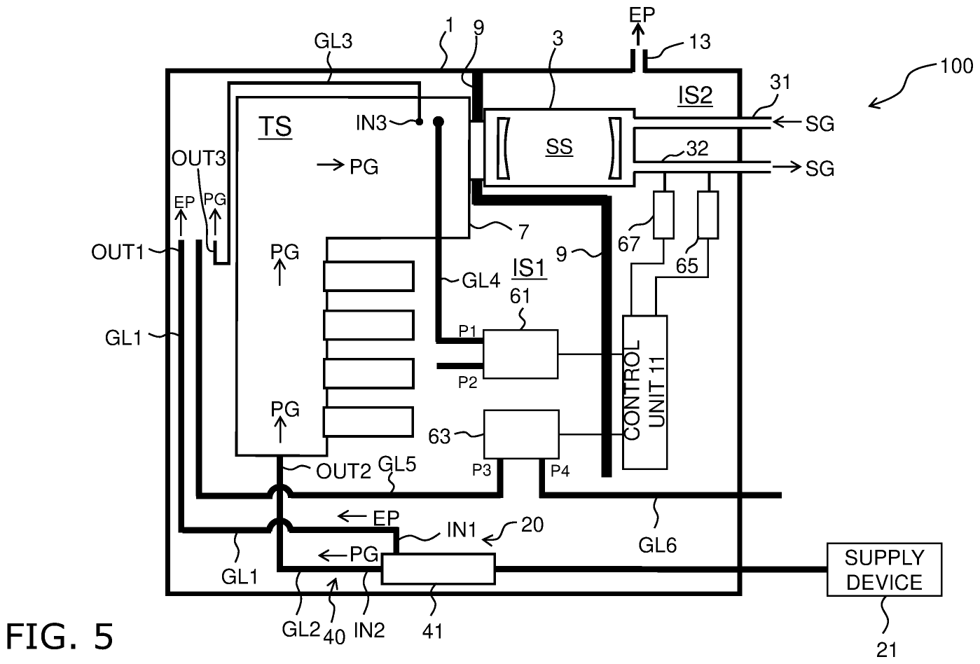

Specifically, as illustrated in FIG. 5, the separating unit 41 is disposed in the housing 1, a remnant gas exhaust port of the separating unit 41 can be the inlet IN1 of the explosion-proof gas introduction unit 20, and a purge gas PG exhaust port can be the inlet IN2 of the purge gas introduction unit 40. FIG. 5 is a diagram illustrating another example of the gas flow structure of the analysis device. With this structure, it is not necessary to individually dispose the gas line for supplying the purge gas PG and the gas line for supplying the explosion-proof gas EP.

### INDUSTRIAL APPLICABILITY

The present invention can be widely applied to analysis devices for analyzing a gas to be measured contained in a sample gas.

1. An analysis device for analyzing a gas to be measured, comprising:
a filling unit configured to be filled with a sample gas containing the gas to be measured;
an irradiation unit configured to emit measurement light to be used for analyzing the gas to be measured;
a propagation unit disposed between the filling unit and the irradiation unit, to form a propagation space that is configured to propagate the measurement light emitted from the irradiation unit to propagate to the filling unit;
a housing configured to house the filling unit, the irradiation unit, and the propagation unit;
a purge gas introduction unit configured to introduce a purge gas into the propagation space; and
an explosion-proof gas introduction unit configured to introduce an explosion-proof gas into the internal space of the housing.

2. The analysis device according to claim 1, further comprising a partition plate configured to separate the internal space of the housing into a first internal space in which the irradiation unit and the propagation unit exist, and a second internal space in which the filling unit exists.

3. The analysis device according to claim 1, wherein the housing and the propagation unit are constituted as an internal pressure explosion-proof container.

4. The analysis device according to claim 1, further comprising a pressure switch configured to detect whether or not a pressure in the filling unit has become equal to or higher than a predetermined pressure lower than a pressure in the internal space of the housing.

5. The analysis device according to claim 1, further comprising a first differential pressure gauge configured to measure a difference between a pressure adjacent to a purge gas outlet in the propagation space and a pressure in the internal space of the housing.

6. The analysis device according to claim 1, further comprising a second differential pressure gauge configured to measure a difference between a pressure adjacent to an explosion-proof gas outlet in the internal space of the housing and a pressure outside the housing.

7. The analysis device according to claim 1, wherein
the propagation unit includes a mirror disposed in the propagation space to guide the measurement light to the filling unit, and
the analysis device further comprises a jig configured to allow a tool that is configured to adjust the mirror to reach a position of the mirror in the propagation space.

8. The analysis device according to claim 1, further comprising a fixing plate configured to fix the filling unit, the irradiation unit, and the propagation unit are fixed.

9. The analysis device according to claim 1, further comprising a separating unit configured to separate the purge gas from a gas.

10. The analysis device according to claim 9, wherein the separating unit is disposed outside the housing.

11. The analysis device according to claim 9, wherein
the separating unit is disposed inside the housing, and
among components generated by separation of the gas using the separating unit, a remnant gas other than the purge gas is used as the explosion-proof gas.

12. The analysis device according to claim 1, further comprising:
a first differential pressure gauge configured to measure a difference between a pressure adjacent to a purge gas outlet in the propagation space and a pressure in the internal space of the housing;
a second differential pressure gauge configured to measure a difference between a pressure adjacent to an explosion-proof gas outlet in the internal space of the housing and a pressure outside the housing; and
a pressure switch configured to detect whether or not a pressure in the filling unit has become equal to or more than a predetermined pressure lower than the pressure in the internal space of the housing, wherein
the first differential pressure gauge, the second differential pressure gauge, and the pressure switch measure a magnitude relationship between the pressure in the internal space of the housing and the pressure outside the housing, a magnitude relationship between the pressure in the propagation space and the pressure in the internal space of the housing, a magnitude relationship between the pressure in the propagation space and the pressure in the internal space of the filling unit, and a magnitude relationship between the pressure in the internal space of the housing and the pressure in the internal space of the filling unit.

13. The analysis device according to claim 1, wherein the gas to be measured is carbon dioxide, carbon monoxide, methane, sulfur dioxide, ammonia, nitrogen oxides, hydrogen chloride, water, ethane, acetylene, propane, ethylene, hexane, propylene, hydrogen sulfide, isobutene, methanol, phosgene, butane, chloroethylene, methyl nitrite, cyclohexane, butadiene, isobutane, isopentane, toluene, hydrogen, hydrogen fluoride, or trifluoropropene.

14. An analysis method of a gas to be measured using an analysis device including a filling unit configured to be filled with a sample gas containing the gas to be measured, an irradiation unit configured to emit measurement light to be used for analyzing the gas to be measured, a propagation unit disposed between the filling unit and the irradiation unit, to form a propagation space that is configured to propagate the measurement light emitted from the irradiation unit to propagate to the filling unit, and a housing configured to house the filling unit, the irradiation unit, and the propagation unit, the method comprising:
introducing a purge gas into the propagation space;
introducing an explosion-proof gas into the internal space of the housing;
emitting the measurement light from the irradiation unit, and propagating the measurement light through the propagation space to the filling unit that is filled with the sample gas; and
analyzing the gas to be measured contained in the sample gas, based on a measurement result of the measurement light after passing through the sample gas filled in the filling unit.
